(19) United States
(12) Patent Application Publication
(10) Pub. No.: US 2021/0293821 A1
(43) Pub. Date: Sep. 23, 2021

(54) METHOD FOR PREDICTING THE OUTCOME OF A TREATMENT WITH AFLIBERCEPT OF A PATIENT SUSPECTED TO SUFFER FROM A CANCER BY MEASURING THE LEVEL OF A PLASMA BIOMARKER

(71) Applicant: SANOFI, Paris (FR)

(72) Inventors: Jennifer DREYMANN, Paris (FR); Caroline PACCARD, Paris (FR); Marielle CHIRON-BLONDEL, Paris (FR)

(21) Appl. No.: 17/185,469

(22) Filed: Feb. 25, 2021

Related U.S. Application Data
(63) Continuation of application No. 15/414,722, filed on Jan. 25, 2017, now abandoned.

(30) Foreign Application Priority Data
Jan. 25, 2016 (EP) 16305065.1

(51) Int. Cl. G01N 33/574; A61P 35/00; A61K 31/4745; A61K 31/513; A61K 31/519; A61K 38/17; C07K 14/71

(52) U.S. Cl. CPC G01N 33/57419; G01N 33/57488; A61P 35/00; A61K 31/4745; A61K 31/513; A61K 31/519; G01N 2333/70542; C07K 14/71; G01N 2800/52; G01N 2333/49; C07K 2319/30; G01N 2333/475; G01N 2333/70525; A61K 38/179

(57) ABSTRACT

The present invention concerns the use of VCAM-1, ICAM-1 and/or PlGF as biomarkers for predicting the outcome of the treatment with aflibercept, or ziv-aflibercept of a patient suspected to suffer from a cancer.

### CROSS-REFERENCE TO RELATED APPLICATIONS

This application is a continuation of U.S. patent application Ser. No. 15/414,722, filed Jan. 25, 2017, which claims priority to European Application No. 16305065.1, filed Jan. 25, 2016, the entire disclosures of which are hereby incorporated herein by reference.

### BACKGROUND OF THE INVENTION

### Field of the Invention

The present invention concerns the use of VCAM-1, ICAM-1 and PlGF as a biomarker for predicting the outcome of the treatment with aflibercept, or ziv-aflibercept, of a patient suspected to suffer from cancer.

Aflibercept, or ziv-aflibercept, also referred to as VEGFR1R2-Fc.DELTA.C1 Flt1D2.Flk1D3.Fc.DELTA.C1 or AVE0005, is a homo dimer protein, with each dimer comprising two identical monomers, each of which is a fusion protein comprising the signal sequence of VEGFR1 fused to the D2 Ig domain of the VEGFR1 receptor, itself fused to the D3 Ig domain of the VEGFR2 receptor, in turn fused to the Fc domain of IgG1.

The protein chain is glycosylated, with N-acetyl-glucosamine, fucose, galactose, mannose and sialic acids contributing to the carbohydrate structures. The N-linked oligosaccharides consist of mainly bi-antennary structures with zero, one or two terminal sialic acids. The monomer has the amino acid sequence SEQ ID N^{o} 1.

The U.S. Food and Drug Administration (FDA) already approved aflibercept under the trade name EYLEA® for the treatment of patients with neovascular (wet) age-related macular degeneration (AMD). In particular, EYLEA® is the trade name for aflibercept as generated, processed and formulated for intravitreal injection.

At the time of registration of aflibercept (ZALTRAP®) for cancer indication, and In light of aflibercept's approved use in treating AMD, the FDA requested that a different name (ziv-aflibercept) be given for the compound's use in the treatment of cancer. Thus, ziv-aflibercept is the United States Adopted Name (USAN) accepted by FDA to designate a pharmaceutical composition comprising aflibercept as generated, processed and formulated for injection via intravenous infusion. Ziv-aflibercept has been approved by the FDA for sale under the tradename ZALTRAP® for the treatment of metastatic colorectal cancer (mCRC).

The European Medicines Agency (EMA) approved ZALTRAP® as well however did not request separate names for the compound. Thus, in the European Union the name “aflibercept” is used regardless of the indication.

ZALTRAP® and EYLEA® are obtained by slightly different processes. They both contain aflibercept or ziv-aflibercept, but the ratio of aggregates of aflibercept or ziv-aflibercept is slightly different in ZALTRAP® and EYLEA®.

ZALTRAP® approval was based on data obtained from the VELOUR trial—a multicenter, randomized, placebo-controlled phase III trial, which compared the efficacy of aflibercept versus placebo in combination with the FOLFIRI regimen for patients with mCRC previously treated with an oxaliplatin containing regimen.

Despites the efficacy and the safety of the treatment of cancer by aflibercept it remains a goal to better identify patients who should benefit more from the treatment.

Indeed the ability to identify Metastatic Colorectal Cancer (mCRC) patients who will benefit from aflibercept would further improve clinical utility of this drug.

To date, no validated predictive serum or plasma biomarkers have been identified that correlate with treatment outcomes to aflibercept.

The profiling of tumor and plasma samples derived from patients involved in clinical trials and subsequent analysis of their genomic/proteomic and clinical data could allow the discovery and potential validation of predictive biomarkers.

It has now been discovered that high levels of VCAM-1, ICAM-1 and/or PlGF at baseline correlated with shorter survival times.

Vascular cell adhesion molecule-1 (VCAM-1) also known as CD106 has the sequence SEQ ID N^{o} 1 (NCBI Reference Sequence: NP_001069.1). The term “VCAM-1” encompasses its homologues and isoforms and variants thereof, as well as fragments of the sequences, provided that the variant proteins (including isoforms), homologous proteins and/or fragments are recognized by one or more VCAM-1 specific antibodies.

Intercellular Adhesion Molecule 1 (ICAM-1) also known as CD54 has the sequence SEQ ID N^{o} 2 (NCBI Reference Sequence: NP_000192.2). The term “ICAM-1” encompasses its homologues and isoforms and variants thereof, as well as fragments of the sequences, provided that the variant proteins (including isoforms), homologous proteins and/or fragments are recognized by one or more ICAM-1 specific antibodies.

“Placental Growth Factor” or “PlGF” comprises the 2 isoforms PlGF1 et PlGF2 which have respectively the sequences SEQ ID N^{o} 3 (NCBI Reference Sequence: NP_002623.2) and SEQ ID N^{o} 4 (NCBI Reference Sequence: NP_001193941.1). The term “PlGF” encompasses PlGF1 et PlGF2 their homologues and isoforms and variants thereof, as well as fragments of the sequences, provided that the variant proteins (including isoforms), homologous proteins and/or fragments are recognized by one or more PlGF specific antibodies.

The correlation of ICAM-1 with patient outcome was tested in clinical trials where patients were treated with bevacizumab.

The correlation of ICAM-1 with patient outcome was found in a trial wherein lung cancer patients were treated with bevacizumab, cisplatin and etoposide (Horn et al, J Clin Oncol 2009; 27:6006-6011). The authors found that “patients who had high ICAM levels had a non significant trend towards improved OS compared with patients who had low levels”, whereas in another study (Dowlati et al, 2008, Clin Cancer Res 14(5), 1407) “Patients with low baseline ICAM had a higher response than those with high ICAM”.

But the correlation of ICAM-1 with patient outcome was not found in colorectal cancer patients treated with bevacizumab, capecitabine and oxaliplatin (Liu et al; Cancer Medicine 2013; 2(2): 234-242).

These articles show the level of unpredictability between various biomarkers studies with the same biologics i.e. bevacizumab.

Furthermore bevacizumab is an anti-VEGF-A antibody. Aflibercept is not an antibody but a chimeric protein. It consists of portions of extracellular domains of human VEGF receptors 1 and 2 fused to human IgG1 Fc portion. Aflibercept binds not only to VEGF-A but also to VEGF-B and placental growth factor (PlGF).

Thus bevacizumab and aflibercept have structure and functions that are very different and the man skilled in the art would not transpose directly the results obtained with bevacizumab to aflibercept.

The correlation of VCAM-1 with patient outcome was found in the two clinical trials mentioned above (Liu et al; Cancer Medicine 2013; 2(2): 234-242 and Horn et al, J Clin Oncol 2009; 27:6006-6011). However in these two studies there is not a placebo arm to let us know if the effect is predictive or prognostic.

### BRIEF SUMMARY OF THE INVENTION

The invention relates to the use of VCAM-1, ICAM-1 and/or PlGF as biomarkers for predicting the outcome of the treatment with aflibercept, or ziv-aflibercept of a patient suspected to suffer from a cancer.

In one aspect, the present invention provides a method of determining whether a patient suspected to suffer from cancer is a candidate for aflibercept, or ziv-aflibercept therapy for the said cancer comprising the step of subjecting a patient's biological sample to at least one assay to measure at baseline the level of a biomarker selected from the group consisting of VCAM-1, ICAM-1 and PlGF, wherein when the biological sample of the biomarker level is low relative to a reference level of expression of the biomarker, the patient is identified as a candidate for therapy for cancer.

In another aspect, the present invention provides a method of determining whether a patient suspected to suffer from cancer is a candidate for aflibercept, or ziv-aflibercept therapy for the said cancer comprising the step of subjecting a patient's biological sample to at least one assay to measure at baseline the level of a biomarker selected from the group consisting of VCAM-1, ICAM-1 and PlGF, wherein when the biological sample level of the biomarker is high relative to a reference level of expression of the biomarker or threshold, the patient is identified as not being a candidate for therapy for cancer. The threshold or reference level of expression of the biomarker allows to define sensitive and non-sensitive populations.

In an embodiment the reference level of expression of VCAM-1 is comprised between around 406 ng/mL and around 577 ng/mL. Yet in another the reference level of expression of VCAM-1 is around 553 ng/mL.

In another embodiment the reference level of expression of ICAM-1 is comprised between around 92 ng/mL and around 145 ng/mL. Yet in another the reference level of expression of ICAM-1 is around 144 ng/mL.

In another embodiment the reference level of expression of PlGF is comprised between around 12 ng/mL and around 19 ng/mL. Yet in another the reference level of expression of PlGF is around 17 ng/mL.

The invention relates also to a method for treating a patient with a cancer with aflibercept, or ziv-aflibercept, comprising administering a therapeutically effective amount of aflibercept, or ziv-aflibercept to the patient, wherein the level of a biomarker selected from the group consisting of VCAM-1, ICAM-1 and PlGF in the patient's biological sample is low relative to a reference level of expression of the biomarker.

Yet in another aspect the invention relates to a method for improving the progression-free survival (PFS) and/or the overall survival (OS) of a patient with a cancer, comprising administering a therapeutically effective amount of aflibercept, or ziv-aflibercept to the patient, wherein the level of a biomarker selected from the group consisting of VCAM-1, ICAM-1 and PlGF in the patient's biological sample is low relative to a reference level of expression of the biomarker.

In an embodiment of one of the methods described above the biological sample is chosen from the group consisting of blood, serum and plasma.

In an embodiment of one of the methods described above the cancer is a colon cancer, a colorectal cancer or a rectal cancer.

In a further embodiment of one of the colorectal cancer is a metastatic colorectal cancer.

In another embodiment of the invention, the subject is treated with aflibercept and further undergoes a chemotherapeutic treatment with oxaliplatin, 5-fluorouracil (5-FU) and folinic acid (i.e. the FOLFOX treatment), folinic acid, 5-fluorouracil and irinotecan (i.e. the FOLFIRI treatment), or 5-fluorouracil and folinic acid (i.e. the FUFOL or LV5FU2 treatment).

The chemotherapeutic treatment may combine at least 2, 3, 4, 5, 6, 7, 8, 9, 10 or at most 10, 9, 8, 7, 6, 5, 4, 3, 2 agents, such as e.g. a combination of oxaliplatin, 5-fluorouracil (5-FU) and folinic acid (i.e. the FOLFOX treatment or the modified FOLFOX6 treatment as described in the example below), a combination of folinic acid, 5-fluorouracil and irinotecan (i.e. the FOLFIRI treatment), or a combination of 5-fluorouracil and folinic acid (i.e. the FUFOL or LV5FU2 treatment).

In this regard the application WO2012146610 relates to a method of treatment of the mCRC by aflibercept, or ziv-aflibercept in combination with FOLFIRI. The content of this application is incorporated by reference.

In an embodiment of one of the methods described above therapeutically effective amounts of aflibercept, or ziv-aflibercept, oxaliplatin, 5-fluorouracil (5-FU) and folinic acid are administered to said patient.

In an embodiment of one of the methods described above therapeutically effective amounts of aflibercept, or ziv-aflibercept, folinic acid, 5-fluorouracil (5-FU) and irinotecan are administered to said patient.

In a further embodiment of one of the methods described above folinic acid at a dosage comprised between about 200 mg/m² and about 600 mg/m², 5-fluorouracil (5-FU) at a dosage comprised between about 2000 mg/m² and about 4000 mg/m², irinotecan at a dosage comprised between about 100 mg/m² and about 300 mg/m² and aflibercept at a dosage comprised between about 1 mg/kg and about 10 mg/kg are administered to patient.

In a further embodiment of one of the methods described above folinic acid at a dosage of about 400 mg/m², 5-fluorouracil (5-FU) at a dosage of about 2800 mg/m², irinotecan at a dosage of about 180 mg/m² and aflibercept at a dosage of about 4 mg/kg are administered to patient.

In a further embodiment of one of the methods described above folinic acid is administered intravenously at a dosage of about 400 mg/m², 5-fluorouracil (5-FU) is administered intravenously at a dosage of about 2800 mg/m², irinotecan is administered intravenously at a dosage of about 180 mg/m² and aflibercept is administered intravenously at a dosage of about 4 mg/kg and wherein the combination is administered every two weeks.

In a further embodiment of one of the methods described above folinic acid, 5-fluorouracil (5-FU), irinotecan and aflibercept are administered intravenously every two weeks for a period comprised between 9 and 18 weeks.

In a further embodiment of one of the methods described above folinic acid is administered intravenously immediately after aflibercept administration. It can be also administered intravenously immediately after aflibercept administration over a period of about 2 hours.

In a further embodiment of one of the methods described above irinotecan is administered intravenously immediately after aflibercept administration. It can be also administered intravenously immediately after aflibercept administration over a period of about 90 minutes.

In a further embodiment of one of the methods described above 5-fluorouracil (5-FU) is administered immediately after aflibercept administration.

In a further embodiment of one of the methods described above a first quantity of 5-fluorouracil (5-FU) is administered intravenously immediately after aflibercept administration and a second quantity of 5-FU is administered intravenously after the first quantity in continuous infusion.

In a further embodiment of one of the methods described above about 400 mg/m² of 5-fluorouracil (5-FU) is administered intravenously over a period of 2 to 4 minutes after aflibercept administration and wherein 2400 mg/m² of 5-FU is administered intravenously over around 46 hours after the administration of the 400 mg/m² in continuous infusion.

In an embodiment said patient has previously been treated with therapy based on oxaliplatin or on bevacizumab.

In another embodiment said patient has failed with chemotherapy, radiotherapy or surgery.

The invention relates also to aflibercept, or ziv-aflibercept for use in treating a patient suspected to suffer from cancer, wherein the patient has been identified as having lower level of a biomarker selected from the group consisting of VCAM-1, ICAM-1 and PlGF in biological sample as compared to the reference level of expression of the biomarker.

The invention further relates to a kit for predicting whether a patient suspected to suffer from cancer is a candidate for aflibercept, or ziv-aflibercept therapy, which kit comprises:
a) means for measuring the level of a biomarker selected from the group consisting of VCAM-1, ICAM-1 and PlGF; and
b) optionally, a label giving instructions for the use of said kit in predicting whether a patient suspected to suffer from cancer is a candidate for aflibercept, or ziv-aflibercept therapy.

Another aspect of the invention further relates to an article of manufacture comprising:
a) a packaging material;
b) means for measuring the level of a biomarker selected from the group consisting of VCAM-1, ICAM-1 and PlGF; and
c) a label giving instructions for the use of said kit in predicting whether a patient suspected to suffer from cancer is a candidate for aflibercept, or ziv-aflibercept therapy.

The above methods and use of the invention may be, for instance, in vitro or ex vivo methods and use.

Means for measuring the expression level of VCAM-1, ICAM-1 and PlGF protein are well-known in the art and include immunoassay such as ELISA assay. For instance the means for measuring VCAM-1 include antibodies specifically binding to VCAM-1.

The level of VCAM-1 protein may be, for instance, determined using immunological detection methods such as an ELISA assay. The methods involve an antibody which binds to VCAM-1 protein, for example a monoclonal or polyclonal antibody, an antibody variant or fragments such as a single chain antibody, a diabody, a minibody, a single chain Fv fragment (sc(Fv)), a Sc(Fv)2 antibody, a Fab fragment or a F(ab′)2 fragment, or a single domain antibody. Such antibodies are well known in the art and are commercially available. They may also notably be obtained by immunization of animals (for example rabbits, rats or mice) with VCAM-1 protein. Antibodies may be used to determine protein expression in a range of immunological assays including competitive and non-competitive assay systems using techniques such as western blotting, immunohistochemistry/immunofluorescence (i.e protein detection on fixed cells or tissues), radioimmunoassay such as RIA (radio-linked immunoassay), ELISA (enzyme linked immunosorbent assay), “sandwich” immunoassays, immunoprecipitation assays, immunodiffusion assays, agglutination assays, complement-fixation assays, immunoradiometric assays, fluorescent immunoassays, e.g. FIA (fluorescence-linked immunoassay), chemiluminescence immunoassays, ECLIA (electrochemiluminescence immunoassay) and protein A immunoassays. Such assays are routine and well known to the person skilled in the art (Ausubel et al (1994) Current Protocols in Molecular Biology, Vol. 1, John Wiley & Sons, Inc., New York).

Protein expression of VCAM-1 may be determined by proteomic method such as mass spectrometry assays (LC-MS or LC-MS/MS). Qualitative and quantitative mass spectrometric techniques are known and used in the art. To this aim, target peptides specific for marker proteins are selected and quantified based on calibration curves established with synthetic peptides labeled with stable isotopes. Enzymatic digests, spiked with a defined amount of isotope labeled target peptides, are analyzed by liquid chromatography coupled with mass spectrometry. The ratio between labeled and non-labeled target peptides is measured to assess target peptide concentrations and therefore protein marker concentration.

The means for measuring the expression level of VCAM-1 may also include reagents such as e.g. reaction and/or washing buffers. The means may be present, e.g., in vials or microtiter plates, or be attached to a solid support such as a microarray as can be the case for primers and probes.

Similar means are at the disposal of the man skilled in the art for detecting ICAM-1 and PlGF.

In an embodiment VCAM-1, ICAM-1 and PlGF proteins can be measured with a bead-based multiplex assay, the Luminex™ technology.

Aflibercept, or ziv-aflibercept is provided in a formulation which is not prejudicial to the patient to be treated.

In an embodiment aflibercept, or ziv-aflibercept is provided in a formulation with sucrose and polysorbate 20 (stabilisers), sodium chloride, citrate buffer, and sodium phosphate buffer, adjusted to final pH.

In another embodiment aflibercept, or ziv-aflibercept, is supplied in two drug product presentations:
a presentation at 100 mg aflibercept, or ziv-aflibercept/4.0 mL (nominal concentration).
a second presentation at 200 mg aflibercept, or ziv-aflibercept/8.0 mL (nominal concentration).

Both presentations are manufactured from the same bulk sterile solution at 25 mg/mL of aflibercept, or ziv-aflibercept.

Prior to infusion to the patient, the concentrate solution is diluted with 0.9% sodium chloride solution or 5% dextrose.

The anti-cancer agents used in the above recited method or use are provided in a pharmaceutically acceptable carrier, excipient or diluent which is not prejudicial to the patient to be treated.

Pharmaceutically acceptable carriers and excipient that may be used in the compositions of this invention include, but are not limited to, ion exchangers, alumina, aluminium stearate, lecithin, self-emulsifying drug delivery systems (SEDDS) such as d-a-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As appreciated by skilled artisans, compositions are suitably formulated to be compatible with the intended route of administration. Examples of suitable routes of administration include parenteral route, including for instance intramuscular, subcutaneous, intravenous, intraperitoneal or local intratumoral injections. The oral route can also be used, provided that the composition is in a form suitable for oral administration, able to protect the active principle from the gastric and intestinal enzymes.

The term “effective amount” refers to an amount of a drug alone or in combination with other drug or treatment regimen effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, duration of progression free survival (PFS), the response rates (RR), duration of response, and/or quality of life.

The terms “Therapy”, “therapeutic”, “treatment” and “treating” are used herein to characterize a therapeutic method or process that is aimed at (1) slowing down or stopping the progression, aggravation, or deterioration of the symptoms of the disease state or condition to which such term applies; (2) alleviating or bringing about ameliorations of the symptoms of the disease state or condition to which such term applies; and/or (3) reversing or curing the disease state or condition to which such term applies.

The term “overall survival (OS)” refers to the length of time during and after treatment the patient survives. As the skilled person will appreciate, a patient's overall survival is improved or enhanced, if the patient belongs to a subgroup of patients that has a statistically significant longer mean survival time as compared to another subgroup of patients.

The term “progression-free survival (PFS)” refers to the length of time during and after treatment during which, according to the assessment of the treating physician or investigator, the patient's disease does not become worse, i.e., does not progress. As the skilled person will appreciate, a patient's progression-free survival is improved or enhanced if the patient belongs to a subgroup of patients that has a longer length of time during which the disease does not progress as compared to the average or mean progression free survival time of a control group of similarly situated patients.

A “subject” or a “patient” may be a human or a non-human mammal, such as monkeys, dogs, cats, guinea pigs, hamsters, rabbits, cows, horses, goats and sheep.

The term “reference level” herein refers to a predetermined value. As the skilled artisan will appreciate the reference level is predetermined and set to meet the requirements in terms of e.g. specificity and/or sensitivity. These requirements can vary, e.g. from regulatory body to regulatory body. It may for example be that assay sensitivity or specificity, respectively, has to be set to certain limits, e.g. 80%, 90% or 95%. These requirements may also be defined in terms of positive or negative predictive values. Nonetheless, based on the teaching given in the present invention it will always be possible to arrive at the reference level meeting those requirements. In one embodiment the reference level is determined in healthy individuals. The reference value in one embodiment has been predetermined in the disease entity to which the patient belongs. In certain embodiments the reference level can e.g. be set to any percentage between 25% and 75% of the overall distribution of the values in a disease entity investigated. In other embodiments the reference level can e.g. be set to the median, tertiles or quartiles as determined from the overall distribution of the values in a disease entity investigated. In one embodiment the reference level is set to the median value as determined from the overall distribution of the values in a disease entity investigated.

### BRIEF DESCRIPTION OF THE DRAWINGS

FIG. 1 represents Kaplan-Meier curves for PFS endpoint for sensitive and non-sensitive populations defined with VCAM-1.

FIG. 2 represents Kaplan-Meier curves for OS endpoint for sensitive and non-sensitive populations defined with VCAM-1.

FIG. 3 represents Kaplan-Meier curves for PFS endpoins for sensitive and non-sensitive populations defined with ICAM-1.

FIG. 4 represents Kaplan-Meier curves for OS endpoint for sensitive and non-sensitive populations defined with ICAM-1.

FIG. 5 represents Kaplan-Meier curves for PFS endpoint for sensitive and non-sensitive populations defined with VCAM-1 and ICAM-1.

FIG. 6 represents Kaplan-Meier curves for OS endpoint for sensitive and non-sensitive populations defined with VCAM-1 and ICAM-1.

FIG. 7 represents Kaplan-Meier curves for PFS endpoint for sensitive and non-sensitive populations defined with PlGF.

### DETAILED DESCRIPTION OF THE INVENTION

### Example: Predictive Effect of VCAM-1, ICAM-1 and/or PlGF on PFS in the AFLAME Study

### Study EFC11338 (Aflame)

EFC11338 was designed as a Multinational, Randomized, Double-Blind Study of Aflibercept Versus Placebo with Irinotecan/5-FU Combination (FOLFIRI) in Patients with Metastatic Colorectal Cancer (MCRC) After Failure of an Oxaliplatin Based Regimen.

### Objectives:

To identify potential predictive biomarkers for response to treatment on efficacy endpoints (Progression Free Survival (PFS), Overall Survival (OS) and Overall Response Rate (ORR))

To identify potential prognostic biomarkers for efficacy endpoints (PFS, OS and ORR)

To identify potential correlation between biomarkers and other baseline characteristics

To identify potential correlation of longitudinal plasma measurements with clinical endpoints

To identify potential groups of homogeneous individuals based on their molecular profiles in an unsupervised way and to estimate correlation with clinical outcomes (PFS, OS and ORR)

To assess the safety profile of the population identified by prognostic/predictive biomarkers

Dosage and Schedule of Administration:

Patients were administered either aflibercept or placebo, depending on arm assigned. Immediately after, patients received irinotecan, 5-FU and leucovorin (FOLFIRI regimen). This treatment was repeated every 2 weeks.

### Aflibercept/Placebo

Arm A, aflibercept: 4 mg/kg was administered IV over 1 hour on Day 1, every 2 weeks, OR

Arm B, placebo: 4 mg/kg was administered IV over 1 hour on Day 1, every 2 weeks.

### FOLFIRI Regimen

Immediately after aflibercept/placebo administration, all the patients received:
Irinotecan 180 mg/m2 IV infusion in 500 mL D5W over 90 minutes and dl leucovorin*400 mg/m2 IV infusion over 2 hours, at the same time, in bags using a Y-line, followed by:
5-FU 400 mg/m2 IV bolus given over 2-4 minutes, followed by:
5-FU 2400 mg/m2 continuous IV infusion in 500 mL D5W (recommended) over 46-hours.

### Duration of Treatment:

Patient was treated until disease progression, unacceptable toxicity or patient's refusal

### Duration of Observation:

Patients were followed when on study treatment and during follow up period until death or the study cut-off date for OS, whichever comes first. The cut-off date for OS is one year after the last patient enrolled.

### Number of Subjects:

Intent-to-Treat (ITT) population: 332 (109 in the placebo group+223 in the aflibercept group)

Evaluable population for response rate: 295 (96 in the placebo group+199 in the aflibercept group)

Evaluable for Luminex biomarkers—Group 1 biomarkers: 295 (99 in the placebo group+196 in the aflibercept group)

Evaluable for Luminex biomarkers—Group 2 biomarkers: 108 (37 in the placebo group+71 in the aflibercept group)

Evaluable for ELISA biomarkers: 329 (107 in the placebo group+222 in the aflibercept group)

Due to an operational error, some patients randomized to the aflibercept arm have received placebo for one or several treatment cycles; or vice versa. A total of 198 patients received at least one misallocated treatment kit.

### Criteria for Evaluation:

### Biomarkers:

Different types of biomarker data were investigated in the current translational research proposal in the AFLAME study:
107 plasma angiogenic factors and inflammatory cytokines measured at baseline, during and after treatment (end of cycle 1 infusion, 48h post aflibercept/placebo on cycle 2 or 3, 30 days after last aflibercept/placebo administration) with Luminex® technology
Free VEGF-A and PlGF measured in plasma samples at baseline with ELISA (Enzyme-Linked Immunosorbent Assay) technology

### Preparation of Samples and Analysis

Plasma biomarkers were measured either with Luminex™ technology (bead-based multiplex assay) or with ELISA.

Plasma Biomarkers Measured with Luminex™ Technology

Plasma angiogenic factors and inflammatory cytokines have been measured at baseline pre cycle 1.

Thirty proteins measured on all the samples were defined as Group 1 biomarkers and 77 proteins measured only for some samples were defined as Group 2 biomarkers. The biomarkers from Group 1 were angiogenic/inflammatory molecules and have been selected based on aflibercept mechanism of action (inhibiting 3 angiogenic factors and their receptors), key candidate biomarkers identified on independent aflibercept studies or literature/experts.

Plasma Biomarkers Measured with ELISA

In addition to plasma biomarkers measured with Luminex™ technology, free VEGF-A and free PlGF concentrations of baseline plasma samples were measured with ELISA technology.

### Results

### Univariate Analysis

Biomarkers have been tested for predictive and prognostic effects for PFS.

VCAM-1 and ICAM-1 have been identified as potentially predictive with corrected Benjamini-Hochberg (BH) p-value 0.2 (295 subjects).

Then sensitive and non-sensitive populations have been defined using VCAM-1 and ICAM-1 (BH p-value 0.2).

PlGF has been identified as potentially predictive by ELISA (unadjusted p-value=0.075).

Identification of sensitive and non-sensitive populations with VCAM-1

The threshold of 6.32 corresponding to 553 ng/mL has been determined to define sensitive (197 individuals) and non-sensitive populations (98 individuals) respectively corresponding to individuals with low values for VCAM-1 and high values for VCAM-1.

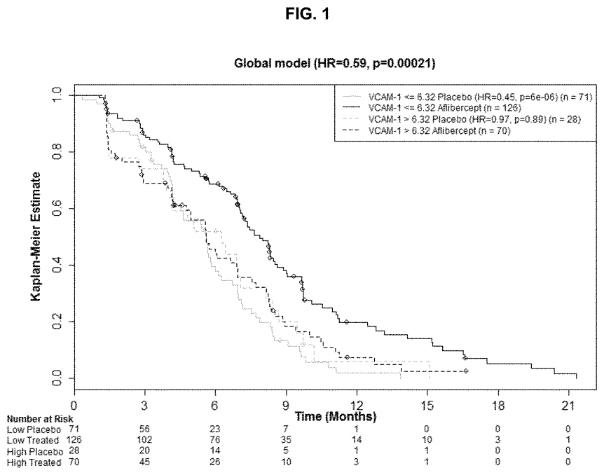

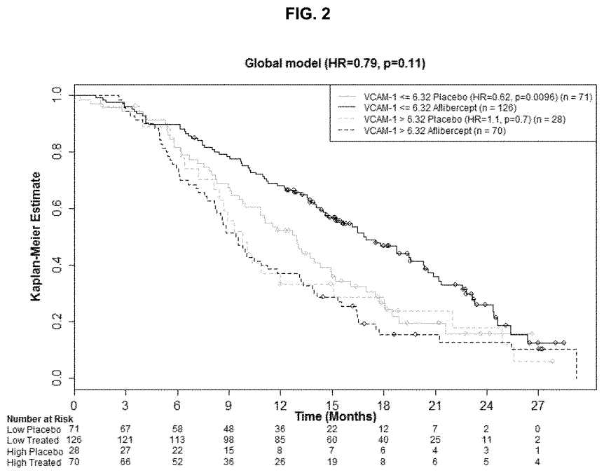

FIG. 1 and FIG. 2 represent Kaplan-Meier curves for PFS and OS endpoints for sensitive and non-sensitive populations illustrating the better treatment effect for low VCAM-1 group compared to high VCAM-1 group. For OS, in addition to a better treatment effect compared to placebo, sensitive population showed globally a better prognostic (increased OS for low VCAM-1 group in placebo and aflibercept arms).

Table 1 shows the response rate for sensitive and non-sensitive populations by treatment group. There was an increased response rate (26%) for the aflibercept/folfiri treatment in sensitive population compared to the non-sensitive population (10%).

TABLE 1
Response Rate for non-sensitive/sensitive populations defined with
VCAM-1 for PFS - Preprocessed data - VCAM-1 and RR
evaluable population

| | Placebo/ | | Aflibercept/ | |
|---|---|---|---|---|
| Population | Folfiri | | Folfiri | |
| Sensitive VCAM-1 <= 6.32 (N = 189) | 3/68 | (4%) | 31/121 | (26%) |
| Non-sensitive VCAM-1 > 6.32 (N = | 1/26 | (4%) | 7/68 | (10%) |
| 94) | | | | |
| Total | 4/94 | (4%) | 38/189 | (20%) |

Identification of Sensitive and Non-Sensitive Populations with ICAM-1

The threshold of 5.04 corresponding to 144 ng/mL has been determined to define sensitive (205 individuals) and non-sensitive populations (90 individuals) respectively corresponding to individuals with low values for ICAM-1 and high values for ICAM-1.

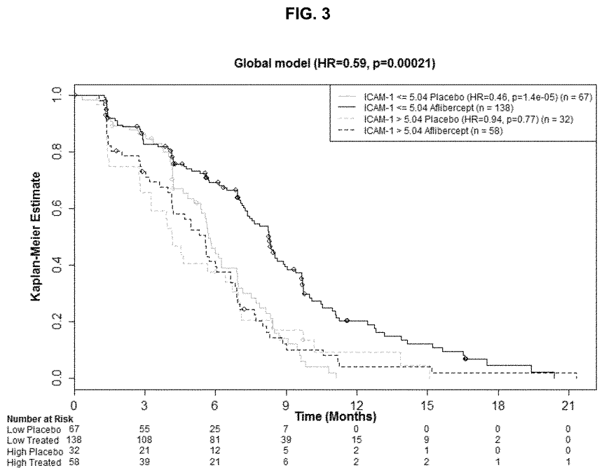

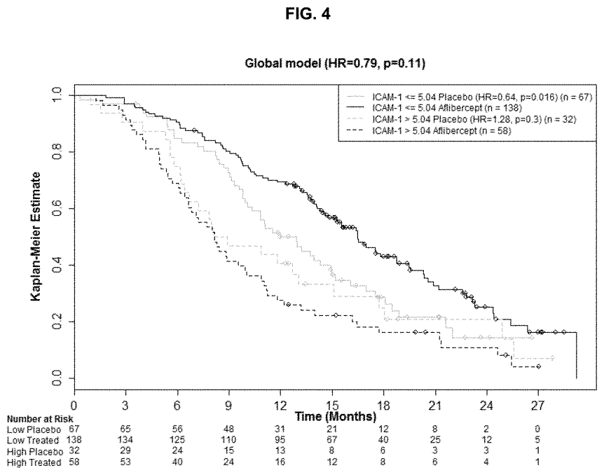

FIG. 3 and FIG. 4 represent Kaplan-Meier curves for PFS and OS endpoints for sensitive and non-sensitive populations illustrating the better treatment effect for low ICAM-1 group compared to high ICAM-1 group. For OS, in addition to a better treatment effect compared to placebo, sensitive population showed globally a better prognostic (increased OS for low ICAM-1 group in placebo and aflibercept arms).

Table 2 shows the response rate for sensitive and non-sensitive populations by treatment group. There has been an increased response rate (25%) for the aflibercept/folfiri treatment in sensitive population compared to the non-sensitive population (9%).

TABLE 2
Response Rate for non-sensitive/sensitive populations defined with
ICAM-1 for PFS - Preprocessed data - ICAM-1 and RR
evaluable population

| | Placebo/ | | Aflibercept/ | |
|---|---|---|---|---|
| Population | Folfiri | | Folfiri | |
| Sensitive ICAM-1 <= 5.04 (N = 197) | 3/63 | (5%) | 33/134 | (25%) |
| Non-sensitive ICAM-1 > 5.04 (N = 86) | 1/31 | (3%) | 5/55 | (9%) |
| Total | 4/94 | (4%) | 38/189 | (20%) |

### Multivariate Analysis

The multivariate predictive score 0.089×ICAM−1+0.17×VCAM−1 has been dichotomized using quantile 10% to 90% as threshold.

Sensitive population showed a significant difference in PFS and in OS in favor of aflibercept over placebo (HR=0.47 for PFS and HR=0.66 for OS), which was increased compared to the non-sensitive population (HR=0.98 for PFS and HR=1.04 for OS).

Median PFS difference between aflibercept and placebo was equal to 2.59 months in sensitive population, showing a greater but moderate difference compared to non-sensitive population (0.5 month).

Median OS difference between aflibercept and placebo was equal to 3.75 months in sensitive population, showing a greater difference compared to non-sensitive population (−0.39 month). For non-sensitive population there was a decrease in median OS for placebo arm and treated arm (8.90 months and 8.51 months) compared to the other populations illustrating a potential prognostic effect of multivariate score in addition to the predictive effect.

In conclusion sensitive population showed a decreased HR compared to the non-sensitive population for PFS and OS with moderate gain in term of median.

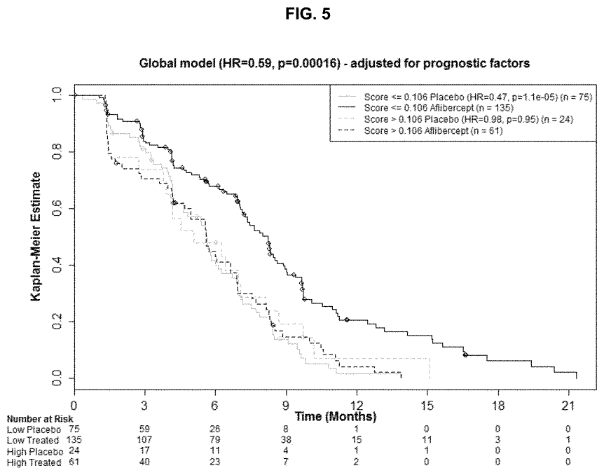

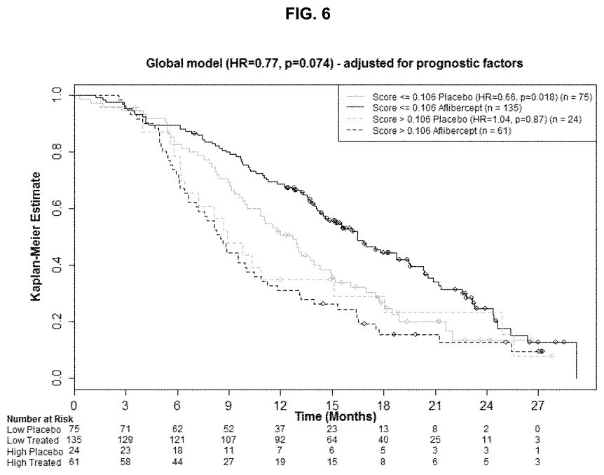

FIG. 5 and FIG. 6 represent Kaplan-Meier curves for PFS and OS endpoints for sensitive and non-sensitive populations illustrating the better treatment effect for low score group compared to high score group. For OS, in addition to a better treatment effect compared to placebo, sensitive population showed globally a better prognostic (increased OS for low score group in placebo and aflibercept arms).

Identification of Sensitive and Non-Sensitive Populations with PlGF

The threshold of 2.82 corresponding to 17 pg/ml has been determined to define sensitive (230 individuals) and non-sensitive populations (99 individuals) respectively corresponding to individuals with low values for PlGF and high values for PlGF.

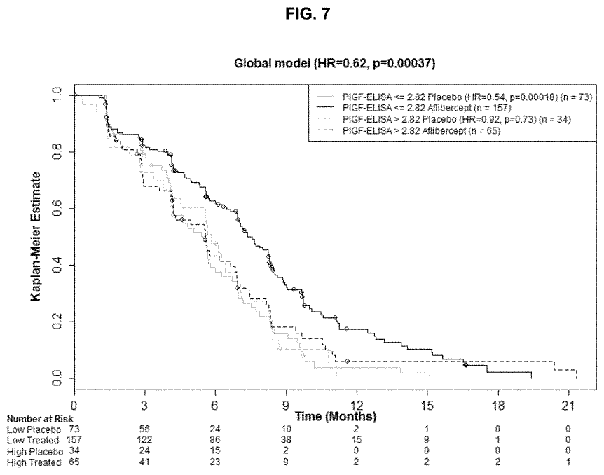

FIG. 7 which represents the Kaplan-Meier curves for PFS endpoint for sensitive and non-sensitive populations defined with PlGF illustrates the determination of the PlGF cut off for sensitive and non-sensitive populations.

### CONCLUSIONS

VCAM-1 and ICAM-1 have been identified as potentially predictive biomarkers for PFS in a univariate framework (unadjusted p-value equal to 0.00017 for VCAM-1 and 0.0043 for ICAM-1).

The third biomarker that showed up to be potentially predictive was PlGF measured by ELISA (unadjusted p-value=0.075).

Linear combination of VCAM-1 and ICAM-1 has been identified as potentially predictive for PFS.

1-3. (canceled)

4. A method for treating a patient a having colon cancer, colorectal cancer or rectal cancer, the method comprising administering a therapeutically effective amount of ziv-aflibercept to the patient,
wherein a level of PlGF detected in a biological sample obtained from the patient is lower than a reference level of expression of PlGF,
optionally wherein the biological sample is selected from the group consisting of a tumor, blood, serum, and plasma.

5-10. (canceled)

11. The method of claim 4, wherein the reference level of expression of PlGF is between about 12 pg/mL and about 19 pg/mL.

12. The method of claim 4, wherein the reference level of expression of PlGF is about 17 pg/mL.

13-14. (canceled)

15. The method of claim 4, wherein the colorectal cancer is a metastatic colorectal cancer.

16. The method of claim 4, wherein the PlGF level of expression which is determined is a circulating level in blood, serum, or plasma.

17. The method of claim 4, further comprising administering to the patient a therapeutically effective amount of folinic acid, 5-fluorouracil (5-FU), and irinotecan.

18. (canceled)

19. A kit for predicting whether a patient suspected to suffer from cancer is a candidate for ziv-aflibercept therapy, wherein the kit comprises:
a) means for measuring the level of PlGF; and
b) optionally, a label giving instructions for the use of the kit in predicting whether a patient suspected to suffer from cancer is a candidate for ziv-aflibercept therapy.

20. An article of manufacture comprising:
a) a packaging material;
b) means for measuring the level of PlGF; and
c) a label giving instructions for the use of the kit in predicting whether a patient suspected to suffer from cancer is a candidate for ziv-aflibercept therapy.

21. The method of claim 4, wherein the ziv-aflibercept is at a dose from about 1 mg/kg to about 10 mg/kg.

22. The method of claim 21, wherein the ziv-aflibercept is at a dose of about 4 mg/kg, and optionally wherein the ziv-aflibercept is administered intravenously.

23. The method of claim 17, wherein the folinic acid is at a dose from about 200 mg/m² to about 600 mg/m².

24. The method of claim 23, wherein the folinic acid is at a dose of about 400 mg/m²; and optionally wherein the folinic acid is administered intravenously.

25. The method of claim 17, wherein the 5-FU is at a dosage from about 2,000 mg/m² to about 4,000 mg/m².

26. The method of claim 25, wherein the 5-FU is at a dose of about 2,800 mg/m²; and optionally wherein the 5-FU is administered intravenously.

27. The method of claim 17, wherein the irinotecan is at a dosage from about 100 mg/m² to about 300 mg/m².

28. The method of claim 27, wherein the irinotecan is at a dose of about 180 mg/m²; and optionally wherein the irinotecan is administered intravenously.


SEQUENCE LISTING (4 sequences)

SEQ ID NO: 1 - protein, 739 aa, Homo sapiens
MPGKMVVILGASNILWIMFAASQAFKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPKNTVISVNPSTKLQEGGSVTMTCSSEGLPAPEIFWSKKLDNGNLQHLSGNATLTLIAMRMEDSGIYVCEGVNLIGKNRKEVELIVQEKPFTVEISPGPRIAAQIGDSVMLTCSVMGCESPSFSWRTQIDSPLSGKVRSEGTNSTLTLSPVSFENEHSYLCTVTCGHKKLEKGIQVELYSFPRDPEIEMSGGLVNGSSVTVSCKVPSVYPLDRLEIELLKGETILENIEFLEDTDMKSLENKSLEMTFIPTIEDTGKALVCQAKLHIDDMEFEPKQRQSTQTLYVNVAPRDTTVLVSPSSILEEGSSVNMTCLSQGFPAPKILWSRQLPNGELQPLSENATLTLISTKMEDSGVYLCEGINQAGRSRKEVELIIQVTPKDIKLTAFPSESVKEGDTVIISCTCGNVPETWIILKKKAETGDTVLKSIDGAYTIRKAQLKDAGVYECESKNKVGSQLRSLTLDVQGRENNKDYFSPELLVLYFASSLIIPAIGMIIYFARKANMKGSYSLVEAQKSKV

SEQ ID NO: 2 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 3 - protein, 170 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERRRPKGRGKRRREKQRPTDCHLCGDAVPRR

SEQ ID NO: 4 - protein, 149 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERCGDAVPRR